SEQUENCE LISTING (4 sequences)

SEQ ID NO: 1 - DNA, 12 bp, Homo sapiens
  misc_feature (4)..(9): n is a, c, g, or t
gcannnnnntgc

SEQ ID NO: 2 - DNA, 13 bp, Homo sapiens
  misc_feature (4)..(10): n is a, c, g, or t
tgannnnnnntca

SEQ ID NO: 3 - DNA, 11 bp, Homo sapiens
  misc_feature (4)..(8): n is a, c, g, or t
gagnnnnnctc

SEQ ID NO: 4 - DNA, 11 bp, Homo sapiens
  misc_feature (4)..(8): n is a, c, g, or t
aagnnnnnctt

(19) United States
(12) Patent Application Publication
(10) Pub. No.: US 2015/0247182 A1
(43) Pub. Date: Sep. 3, 2015

(54) SINGLE CELL ANALYSIS USING SEQUENCE TAGS

(71) Applicant: ADAPTIVE BIOTECHNOLOGIES CORP., Seattle, WA (US)

(72) Inventors: Malek Faham, Seattle, WA (US); Thomas Willis, Seattle, WA (US); Jianbiao Zheng, Seattle, WA (US)

(73) Assignee: ADAPTIVE BIOTECHNOLOGIES CORP., Seattle, WA (US)

(21) Appl. No.: 14/425,036

(22) PCT Filed: Jul. 22, 2013
(86) PCT No.: PCT/US2013/051539

§ 371 Date: Feb. 28, 2015

Related U.S. Application Data
(60) Provisional application No. 61/675,254, filed on Jul. 24, 2012.

(51) Int. Cl. C12Q 1/68

(52) U.S. Cl. CPC C12Q 1/6806

(57) ABSTRACT

The invention provides a method of making measurements on individual cells of a population by forming reactors containing single cells and a predetermined number, usually one, homogeneous sequence tag. In one aspect, the invention provides a method of making multiparameter measurements on individual cells of such a population by carrying out a polymerase cycling assembly (PCA) reaction to link their identifying nucleic acid sequences, such as sequence tag copies derived from a homogeneous sequence tag, to other cellular nucleic acids of interest, thereby forming fusion products. The fusion products of such PCA reactions are then sequenced and tabulated to generate multiparameter data for cells of the population.

### CROSS-REFERENCE

The application claims the benefit of U.S. Provisional Patent Application No. 61/675,254, filed Jul. 24, 2012, which is incorporated by reference in its entirety.

### BACKGROUND

Cytometry plays an indispensable role in many medical and research fields. Image-based and flow cytometers have found widespread use in these fields for counting cells and measuring their physical and molecular characteristics, e.g. Shapiro, Practical Flow Cytometry, 4th Edition (Wiley-Liss, 2003). In particular, flow cytometry is a powerful technique for rapidly measuring multiple parameters on large numbers of individual cells of a population enabling acquisition of statistically reliable information about the population and its subpopulations. The technique has been important in the detection and management of a range of diseases, particularly blood-related diseases, such as hematopoietic cancers, HIV, and the like, e.g. Woijciech, Flow Cytometry in Neoplastic Hematology, Second Edition (Informa Healthcare, 2010); Brown et al, Clinical Chemistry, 46: 8(B): 1221-1229 (2000). Despite this utility, flow cytometry has a number of drawbacks, including limited sensitivity in rare cell detection, e.g. Campana et al, Hematol. Oncol. Clin. North Am., 23(5): 1083-1098 (2009); limitations in the number of cell parameters that can be practically measured at the same time; and costly instrumentation.

In view of the above, it would be advantageous to many medical and research fields if there were available alternative methods and systems for making multiparameter measurements on large numbers of individual cells that overcame the drawbacks of current cytometric approaches.

### SUMMARY OF THE INVENTION

The present invention is directed to methods for making multiparameter measurements of target nucleic acids of individual cells of a population by generating for each cell one or more fusion products of such nucleic acids and a unique sequence tag. Aspects of the present invention are exemplified in a number of implementations and applications, some of which are summarized below and throughout the specification.

In one aspect, the invention includes a method of analyzing a plurality of target nucleic acids of single cells of a population comprising the steps of: (a) providing multiple reactors each containing a single cell of the population and a single homogeneous sequence tag in an amplification mixture, the amplification mixture comprising a pair of primers for amplifying each target nucleic acid of the plurality; (b) providing amplifiable sequence tags from the homogeneous sequence tags; (c) amplifying the target nucleic acids and amplifiable sequence tags to form amplicons comprising sequence tags; and (d) sequencing the amplicons from the reactors to identify the target nucleic acids of each cell from the population by the sequence tags incorporated into the amplicons. In some embodiments, the method further comprises a step of lysing the single cells in the reactors prior to the step of amplifying. In further embodiments, reactors are water-in-oil micelles made by a microfluidics device. In still further embodiments, micelles of the invention have a uniform size distribution; for example, in some embodiments, micelles have a distribution of volumes with a coefficient of variation of thirty percent or less.

These above-characterized aspects, as well as other aspects, of the present invention are exemplified in a number of illustrated implementations and applications, some of which are shown in the figures and characterized in the claims section that follows. However, the above summary is not intended to describe each illustrated embodiment or every implementation of the present invention.

### BRIEF DESCRIPTIONS OF THE DRAWINGS

FIG. 1A illustrates steps of one embodiment of the method of the invention.

FIG. 1B illustrates data from single cell analysis from one embodiment of the invention.

FIGS. 1C-1F illustrate various embodiments of homogeneous sequence tags.

FIG. 1G illustrates an enzymatic method of releasing sequence tagged primers from a homogeneous sequence tag in a bead format.

FIG. 1H illustrates a method of attaching sequence tagged primer binding sites to target nucleic acids using a ligase and flap endonuclease.

FIG. 1I illustrates components of a reaction illustrated in FIG. 1H.

FIG. 1J illustrates an embodiment in which a unique sequence tag is attached to each end of target polynucleotides.

FIG. 1K diagrammatically illustrates a microfluidics device for enriching micelles containing both a cell and a homogeneous sequence tag.

FIGS. 2A-2C illustrate a PCA scheme for linking target sequences where pairs of internal primers have complementary tails.

FIGS. 3A-3C illustrate a PCA scheme for linking target sequences where only one primer of each pair of internal primers has a tail that is complementary to an end of a target sequence.

FIGS. 4A-4C illustrate a PCA scheme for linking target sequences where pairs of internal primers have complementary tails and external primers have tails for continued amplification of an assembled product by PCR.

FIGS. 5A-5F illustrate a multiplex of pairwise assemblies of target sequences.

FIGS. 6A-6E illustrate a method of using PCA to link together three sequences.

FIG. 7 illustrates an embodiment for providing a homogeneous sequence tag from a random segment of a cell's genomic DNA.

### DETAILED DESCRIPTION OF THE INVENTION

The practice of the present invention may employ, unless otherwise indicated, conventional techniques and descriptions of organic chemistry, molecular biology (including recombinant techniques), cell biology, and biochemistry, which are within the skill of the art. Such conventional techniques include, but are not limited to, sampling and analysis of blood cells, nucleic acid sequencing and analysis, and the like. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV); PCR Primer: A Laboratory Manual; and Molecular Cloning: A Laboratory Manual (all from Cold Spring Harbor Laboratory Press); Ausubel, editor, Current Protocols in Molecular Biology (John Wiley & Sons, electronic and print editions); and the like.

The invention provides methods for analyzing multiple nucleic acids in individual cells or particles of a population. In one aspect, a reaction is carried out on the nucleic acids of each individual cell or particle to link a unique sequence tag to one or more cellular nucleic acids of interest, after which conjugates of the sequence tags and target nucleic acids (referred to herein as “fusion products”) are analyzed by high throughput nucleic acid sequencing. That is, each cell or particle whose nucleic acids are analyzed receives a unique sequence tag by which nucleic acids from it may be identified and from which nucleic acids from other cells may be distinguished. The products of such linking, i.e. the conjugates mentioned above, are referred to herein as “fusion products.” After their generation, fusion products are sequenced and tabulated to generate data, especially multiparameter data, for each cell or particle of a population. Such data may include gene expression data, data on the presence or absence of one or more predetermined genomic sequences (such as cancer genes), gene copy number data, or combinations of the foregoing. In some embodiments, such data particularly comprises gene expression data, such as derived from messenger RNA extracted from the cytoplasm of cells. Cells analyzed may include blood cells, cells disaggregated from tissue, single-cell organisms, circulating tumor cells, or the like. Particles analyzed may include organelles, exosomes, vesicles, microvesicles, or the like. In one embodiment, cells and/or particles to be analyzed are from the same sample or the same biological source, such as (for example) a tissue sample of a patient. In other embodiments, cells and/or particles to be analyzed may be mixtures of samples or from multiple biological sources. In some embodiments, cells analyzed by methods of the invention lack cell walls. In other embodiments, cells analyzed by methods of the invention are mammalian cells, and more particularly, human cells.

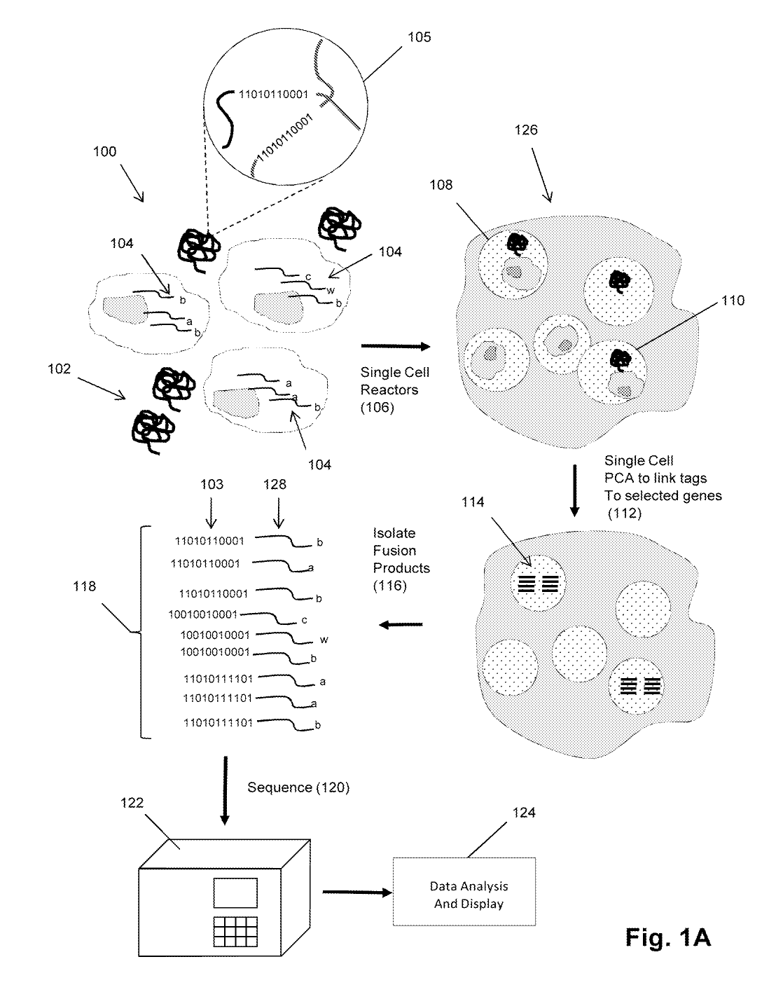

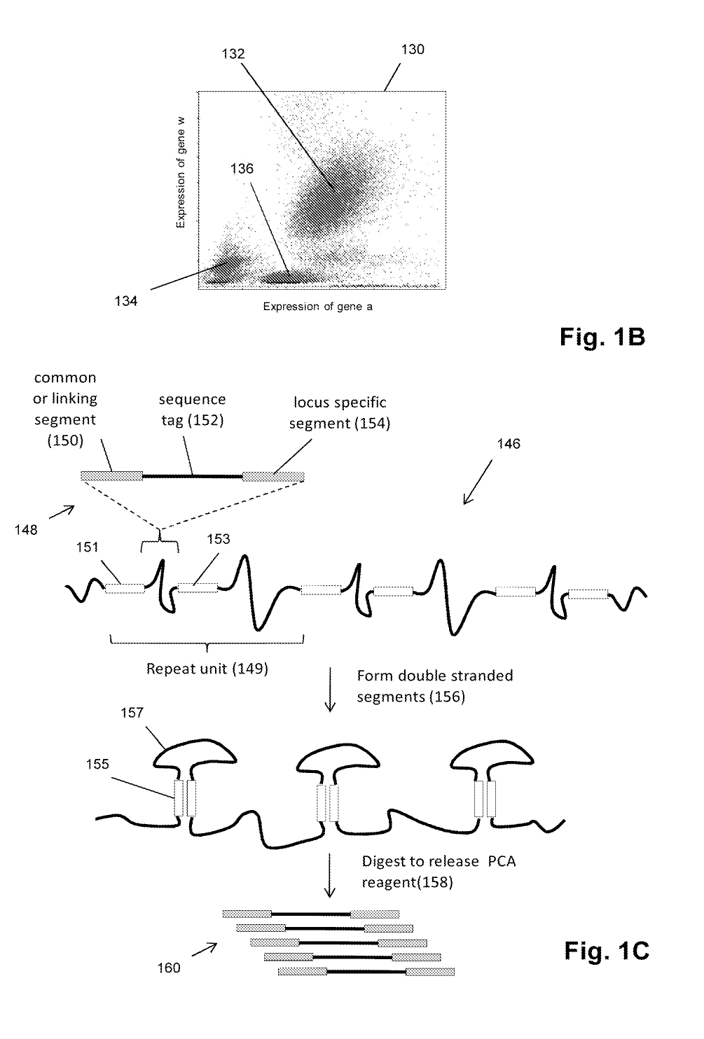

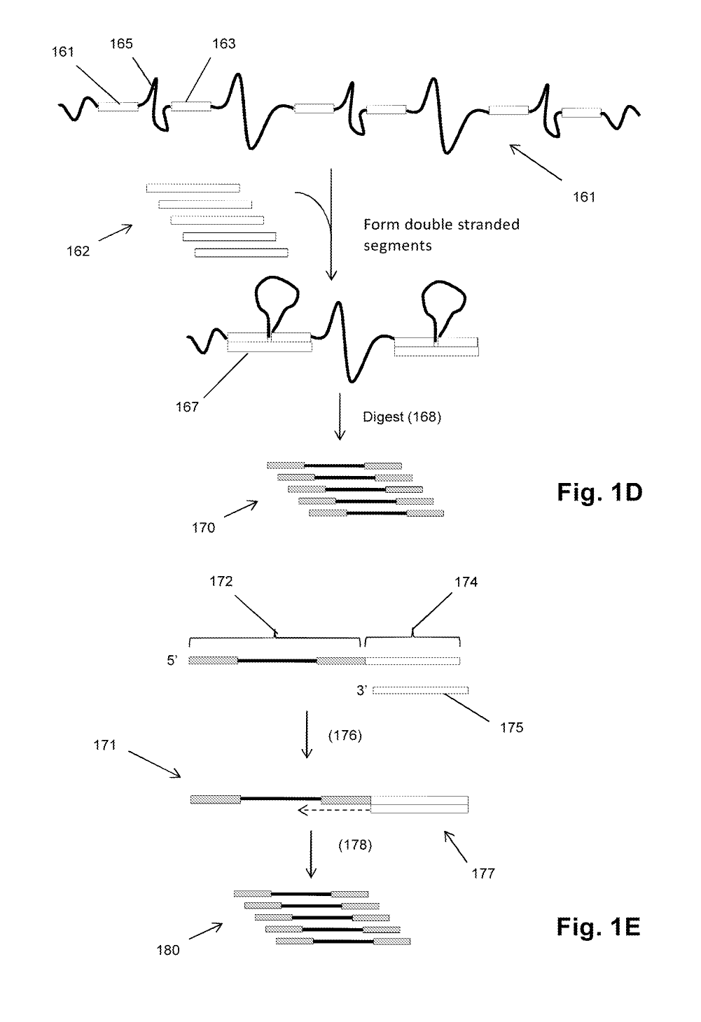

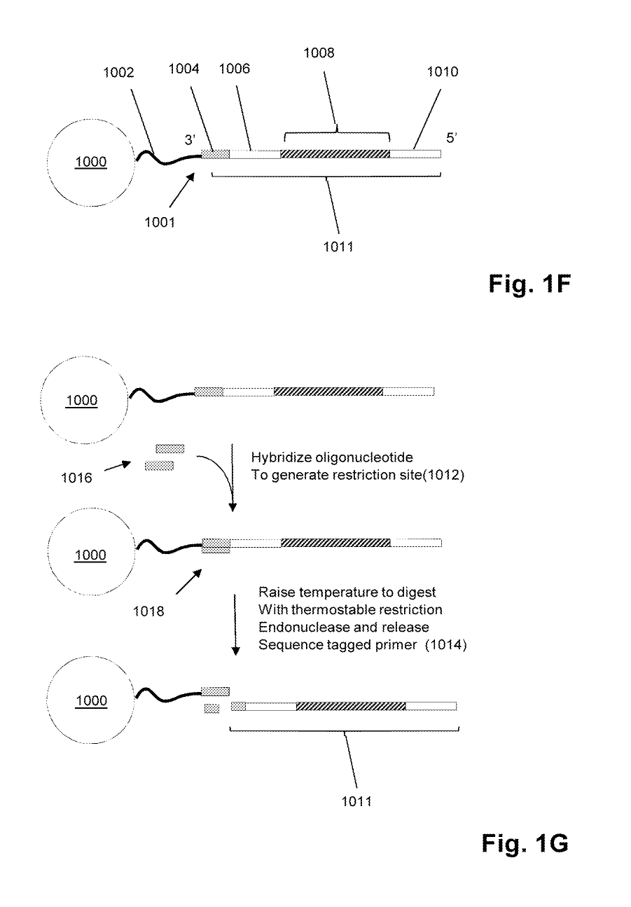

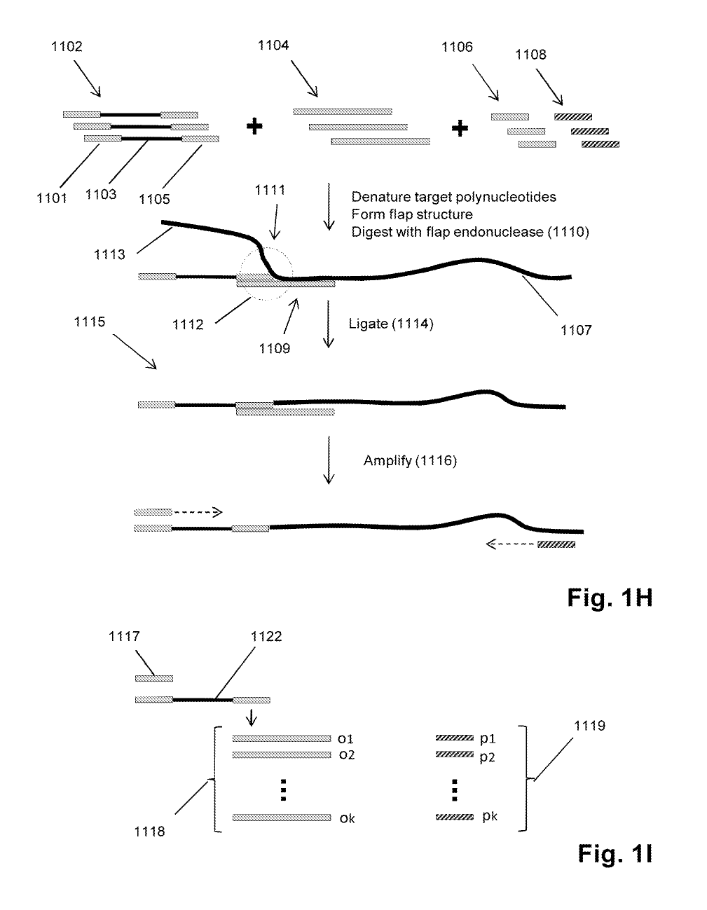

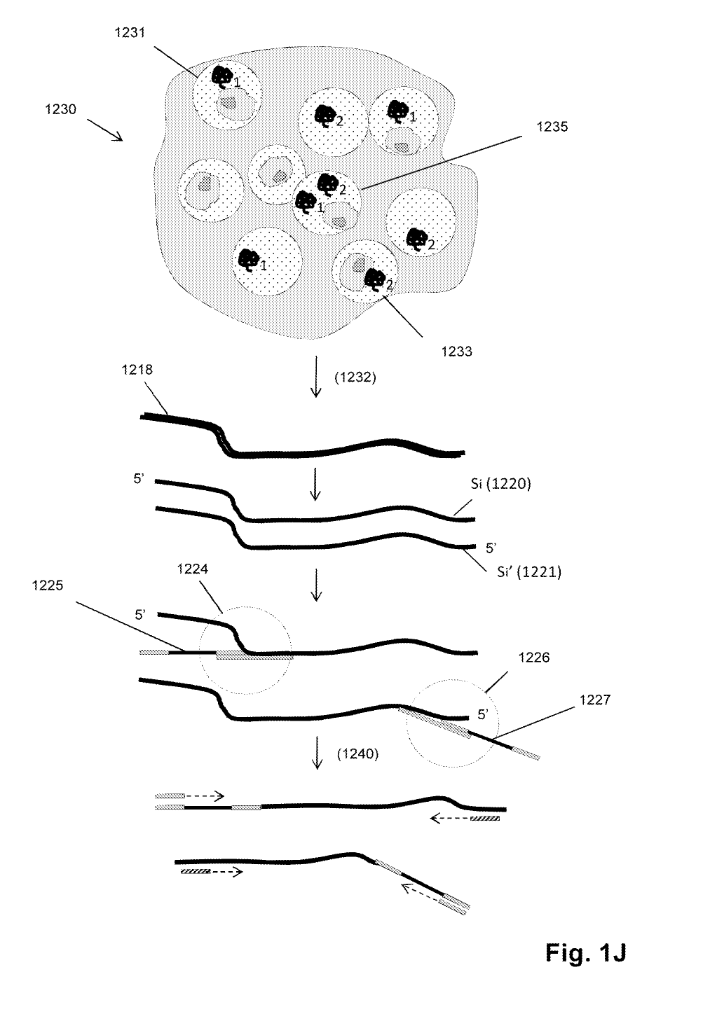

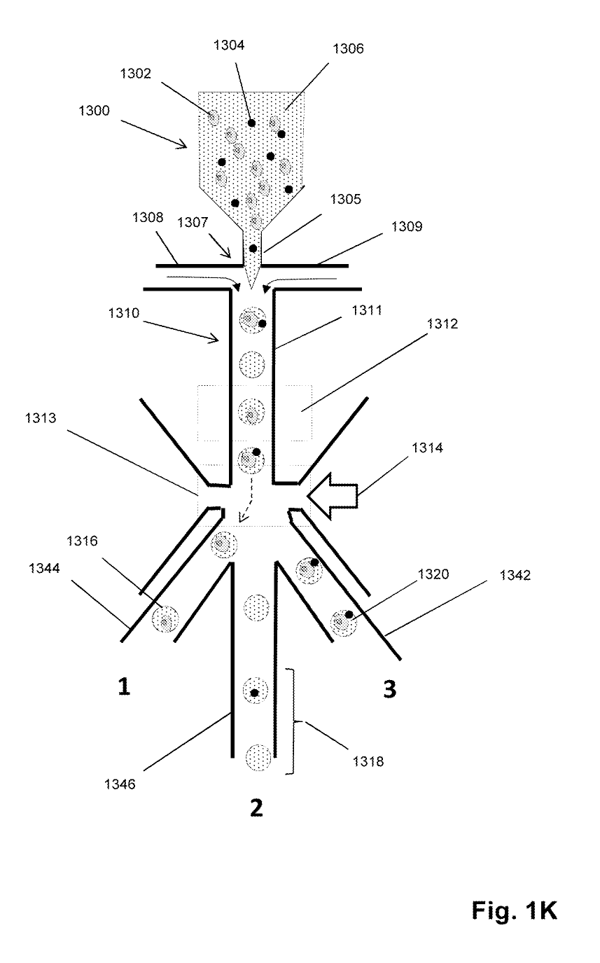

In some embodiments, a single sequence tag is attached to multiple target nucleic acids by a polymerase cycling assembly (PCA) reaction. In other embodiments, one sequence tag is attached to each target nucleic acid. FIG. 1A gives an overview on one embodiment of the invention. Cells (100) are combined with homogeneous sequence tags (102) in a PCA reaction mixture, after which the PCA reaction mixture is partitioned into small reaction volumes, so that a number of such volumes each contain a single cell and a single homogeneous sequence tag. Such partitioning may be carried out in a variety of ways disclosed more fully below. In some embodiments, partitioning is accomplished by generating a water-in-oil emulsion (126) in which micelles, such as (110), serve as single cell reactors. A portion of micelles, such as micelles (108) and (110), contain a single cell and a single homogeneous sequence tag. In such micelles, target nucleic acids are uniquely labeled by the homogeneous sequence tag. As discussed more fully below, homogeneous sequence tags may have a variety of formats. In the embodiment of FIG. 1A, homogeneous sequence tags (102) are products of rolling circle amplification reactions, i.e. RCA amplicons, which comprise copies of a sequence tagged primer. Blow-up (105) represents sequence tags as binary numbers in a single stranded RCA amplicon. In one embodiment, such sequence tagged primers are linear oligonucleotides each comprising a primer binding site at its 5′ end, a target specific sequence at its 3′ end, and a sequence tag sandwiched in between (e.g. illustrated as one embodiment in FIG. 1C). Such PCA reagent may be an inside primer or outside primer in a PCA reaction. In another embodiment, instead of being primers, the sequence tag-containing elements of homogeneous sequence tag (102) may be treated as a target nucleic acid in a PCA reaction. That is, instead of segment (154) being locus specific, it may also be specific for a common or linking primer, so that it is amplified along with cellular target nucleic acids in a PCA reaction to result in a fusion product containing at least one sequence tag.

Each cell has and/or expresses various nucleic acids of interest (104), that is, target nucleic acids, represented by the letters “a”, “b”, “c” and “w”, which may be genomic DNA, RNA, expressed genes, or the like. RNA target nucleic acids are typically converted into DNA by a reverse transcriptase reaction using conventional reagents and techniques, e.g. as disclosed in Tecott et al, U.S. Pat. No. 5,168,038. In accordance with the invention, cells (100) are disposed (106) in single cell reactors, which in this example are illustrated as micelles of a water-in-oil emulsion (126), although a variety of single cell reactors may be used, including but not limited to, plates with arrays of nanoliter-volume wells, microfluidic devices, and the like, as described more fully below. In one aspect, single-cell emulsion (126) is generated using a microfluidic emulsion generator, such as disclosed by Zeng et al, Anal. Chem., 82: 3183-3190 (2010), or the like.

Single cell reactors (such as the micelles of emulsion (126)) contain a PCA reaction mixture that, for example, may comprise a nucleic acid polymerase, outer primers and linking primers (described more fully below), nucleoside triphosphates, a buffer solution, and the like. In some embodiments, a PCA reaction mixture may also include one or more cell lysing reagents, so such reagents can more readily gain access to target nucleic acids. For each reactor, e.g. (110), containing a cell and a homogeneous sequence tag, PCA reaction (112) generates fusion products (114) that may comprise one or more pairs of sequences, such that one member of the pair is a sequence tag and the other member is a nucleic acid of interest, such as an expressed gene, a cancer gene, or the like. In other embodiments, fusion products may comprise triplets of sequences, or higher order concatenations. In some embodiments, a single kind of fusion product may be generated for each cell (or per reactor) or a plurality of different kinds of fusion products may be generated for each cell (or per reactor). Such plurality may be in the range of from 2 to 1000, or from 2 to 200, or from 2 to 100, or from 2 to 20. In one embodiment, such plurality may be in the range of from 2 to 10. It is understood that in some embodiments, at least one sequence tag is included within such pluralities.

After completion of PCA reaction (112), emulsion (126) is broken and fusion products (114) are isolated (116). Fusion products (114) are represented in FIG. 1 as conjugates (118) of sequence tags (103) and target nucleic acids (128). A variety of conventional methods may be used to isolate fusion products (114), including, but not limited to, column chromatography, ethanol precipitation, affinity purification after use of biotinylated primers, gel electrophoresis, or the like. As part of PCA reaction (112) or after isolation (116), additional sequences may be added to fusion products (114) as necessary for sequencing (120), for example, using P5 and P7 primers for Illumina-based sequencing. Sequencing may be carried out using a conventional high-throughput instrument (122), e.g. Genome Analyzer IIx (Illumina, Inc., San Diego), or the like. Data from instrument (122) may be analyzed and displayed (124) in a variety of ways. In one embodiment, where target nucleic acids are selected gene expression products, e.g. mRNAs, plots may be constructed that display per-cell expression levels of selected gene for an entire population or subpopulation, in a manner similar to that for flow cytometry data, as illustrated by plot (130). Each cell is associated with a unique sequence tag that is linked via the PCA reaction to genes expressed in the cell in a proportion related to their cellular abundance Thus, by counting the number of expressed gene sequences linked to a specific clonotype sequence, one obtains a measure of expression for such gene in the cell associated with the specific sequence tag. As illustrated in plot (130) of FIG. 1B, three subpopulations of cells are indicated by the presence of separate clusters (132, 134, and 136) based on expression levels of gene w and gene a. In some embodiments, whenever gene expression levels are monitored, at least one gene is selected as an internal standard for normalizing the expression measurements of other genes.

### Homogeneous Sequence Tags for Partitioned Cell Samples

A homogeneous sequence tag is a reagent that comprises a plurality of identical sequence tags or that is capable of generating a plurality of identical sequence tags under defined reaction conditions. Homogeneous sequence tags may have a variety of formats including, but not limited to, (i) rolling circle amplification (RCA) amplicon containing repeated copies of the same sequence tag, (ii) bead-anchored sequence tags, (iii) self-reproducing sequence tags, and the like. A common property of homogeneous sequence tags is that such a tag comprises a single molecular or particulate entity that is capable of releasing or producing multiple copies of the same sequence tag. Homogeneous sequence tags are useful for producing reactors containing a single cell and a unique reagent (e.g. a sequence-tagged primer for a PCR or PCA reaction). This condition may be achieved by appropriately adjusting concentrations of cells and homogeneous sequence tags in a reaction mixture and partitioning the reaction mixture into small volumes so that a portion of such volumes each contains a single cell and a single homogeneous sequence tag. In some embodiments, this is accomplished by forming aqueous micelles in a water-in-oil emulsion, as described more fully below. In some embodiments, multiple homogeneous sequence tag formats may be employed together.

FIGS. 1C and 1D show two exemplary homogeneous sequence tags based on RCA amplicons. In both examples the end reagent released by the homogeneous sequence tag is a sequence tagged-primer for use in a PCA reaction. In FIG. 1C, RCA amplicon (146) is produced using conventional techniques, e.g. Fire et al, U.S. Pat. No. 5,648,245 (which is incorporated by reference) and is designed to include repeat unit (149) which, in turn, includes sequence tagged primer (148) and reverse complementary stem segments (151) and (153). In some embodiments, sequence tagged primer (148) comprises three segments: (i) a 5′ segment (150) that either comprises a linking sequence (as described below for linking target polynucleotides if it is an inner primer in a PCA) or a common primer sequence (for example, if it is an outer primer in a PCA), (ii) sequence tag (152), and (iii) a locus specific segment or primer for annealing to a target polynucleotide so that polymerase extension can occur. After creation of RCA amplicon (146), conditions are adjusted so that stem segments (151) and (153) form double stranded stems (155) that contain restriction endonuclease recognition sites for cleaving RCA amplicon (146), thereby releasing sequence tagged primers in loops (157). So that digestion does not commence upon combining the RCA amplicon with a restriction endonuclease, the latter may be selected from thermostable restriction endonucleases or nickases, so that the reagents may be combined at a lower temperature, e.g. room temperature, and cleavage may be initiated by raising the temperature to the optimal cleavage temperature of the enzyme. Exemplary thermostable restriction endonucleases include Bsp QI (available from New England Biolabs). After cleavage (158), sequence tagged primers (160) are released.

In FIG. 1D, RCA amplicon (161) is generated using conventional techniques. Segments (161) and (163) sandwich sequence tagged primer (165). Upon addition of oligonucleotides (162) containing regions complementary to segments (161) and (163), duplexes (167) form which contain restriction endonuclease sites. Restriction endonucleases and site positions are selected so that upon cleavage (168) sequence tagged primers (170) are released. As above, thermostable restriction endonucleases and/or nickases may be used so that the RCA amplicon and enzymes may be combined at a lower temperature with no digestion (for example, during emulsion preparation) and then the temperature may be increased to initiate digestion and release of the sequence tagged primers (for example, within micelles of an emulsion).

In FIG. 1E, a homogeneous sequence tag comprises a nucleic acid structure that generates sequence tagged primers in a combined polymerase extension reaction and nickase reaction (an isothermal exponential amplification reaction, or EXPAR). EXPARs are disclosed in Van Ness et al, U.S. Pat. No. 7,112,423, which is incorporated by reference. EXPAR nucleic acid structure (171) comprises a double stranded DNA portion (177) (formed by annealing oligonucleotide (175) to segment (174)) and single stranded portion (172) which serves as a template for polymerase extensions from the 3′ end of (175). Within double stranded portion (177) there is a nickase site positioned so that it nicks the polymerase extension at the boundary between segments (172) and (174). Thus, with polymerase and nickase activities present with dNTPs in an appropriate buffer (178), sequence tagged primers (180) are continuously generated.

Homogeneous sequence tags may also be bead-based, as illustrated in FIGS. 1F and 1G. In this embodiment, identical sequence tagged primers are synthesized on beads so that they may be chemically or enzymatically released after single cell reactors are formed. In one aspect, sequence tagged primers are chemically synthesized on beads using a conventional chemistry, e.g. phosphoramidite chemistry. Beads with identical (i.e. clonal) populations of sequence tags are produced by conventional split and mix synthesis of the sequence tag portion of the sequence tagged primers, e.g. Yang et al, Nucleic Acids Research, 30(23): e132 (2002). FIG. 1F illustrates one embodiment of a chemically synthesized homogeneous sequence tag. In the figure, only one strand is shown attached to solid support (1000) for clarity, but a fully loaded bead is understood. The size and composition of solid support (1000) and the selection of linker (1002) are design choices depending in part on the application. In this embodiment, sequence tagged primer (1011) comprises the following elements starting from a 3′ end (1001) proximal to solid support (1000): segment (1004) containing one strand of a restriction endonuclease site; segment (1006) that comprises a primer specific for a target nucleic acid; sequence tag (1008); and segment (1010) comprising a primer binding site for a common primer for amplifying the tagged target polynucleotides. As shown in FIG. 1G, in one embodiment, oligonucleotide (1016) complementary to segment (1004) is combined (1012) with solid supports (1000) in a reaction mixture prior to distribution to reactors under conditions that permit duplexes (1018) to form. Duplex (1018) contains a restriction site for a restriction endonuclease that is activated upon raising temperature. It is clear to one of ordinary skill that the sequence composition and length of duplex (1018) depends of the operating temperature of a thermostable restriction endonuclease used to cleave sequence tagged primers (1011) from solid support (1000). Upon increasing temperature (1014) to activate the restriction enzyme, attached sequence tagged primers (1011) with duplexes (1018) are cleaved from solid support (1000), thereby releasing operable sequence tagged primers (1011). Depending on the cleavage characteristics of the restriction endonuclease, the 3′ end of sequence tagged primer (1011) may be selected to be complementary to a target polynucleotide (for example, type IIs enzyme Bsp QI permit such selection). For other restriction enzymes, the 3′ end of sequence tagged primer may be specific for the 5′ tail of an adaptor primer that is, in turn, specific for a target nucleic acid.

### Polymerase Cycling Assembly (PCA) Reaction Formats

Polymerase cycling assembly (PCA) reactions (also sometimes referred to as linking PCRs) permit a plurality of nucleic acid fragments to be fused together to form a single fusion product in one or more cycles of fragment annealing and polymerase extension, e.g. Xiong et al, FEBS Micro biol. Rev., 32: 522-540 (2008). PCA reactions come in many formats. In one format of interest, PCA comprises a plurality of polymerase chain reactions (PCRs) taking place in a common reaction volume, wherein each component PCR includes at least one linking primer that permits strands from the resulting amplicon to anneal to strands from another amplicon in the reaction and to be extended to form a fusion product or a precursor of a fusion product. PCA in its various formats (and under various alternative names) is a well-known method for fragment assembly and gene synthesis, several forms of which are disclosed below and in the following references, which are incorporated by reference: Yon et al, Nucleic Acids Research, 17: 4895 (1989); Stemmer et al, U.S. Pat. No. 5,928,905; Chen et al, J. Am. Chem. Soc., 116: 8799-8800 (1994); Stemmer et al, Gene, 164: 49-53 (1995); Hoover et al, Nucleic Acids Research, 30 (10): e43 (2002); Xiong et al, Biotechnology Advances, 26: 121-134 (2008); Xiong et al, FEBS Microbiol. Rev., 32: 522-540 (2008); and the like.

Specific PCA reaction conditions may vary widely for particular embodiments and may include routine design choices for those of ordinary skill in the art. Exemplary PCA reaction conditions may comprise the following: 39.4 μL distilled water combined with 10 μL of 10× buffer (100 mM Tris-HCl, pH 8.3, 500 mM KCl, 15 mM MgCl2, and 0.01% gelatin), 2 μL of a 10 mM solution of each of the dNTPs, 0.5 μL of Taq polymerase (5 units/μL), 1 μL of each outer primer (from a 100 μM stock solution) and 10 μL of each inner primer (from a 0.1 μM stock solution). Typically, in PCA reactions the concentrations of outer primers are greater than the concentrations of inner primers so that amplification of the fusion product continues after initial formation. For example, in one embodiment for fusing two target nucleic acids outer primer concentration may be from about 10 to 100 times that of the inner primers, e.g. 1 μM for outer primers and 0.01 μM for inner primers. Otherwise, a PCA reaction may comprise the components of a PCR.

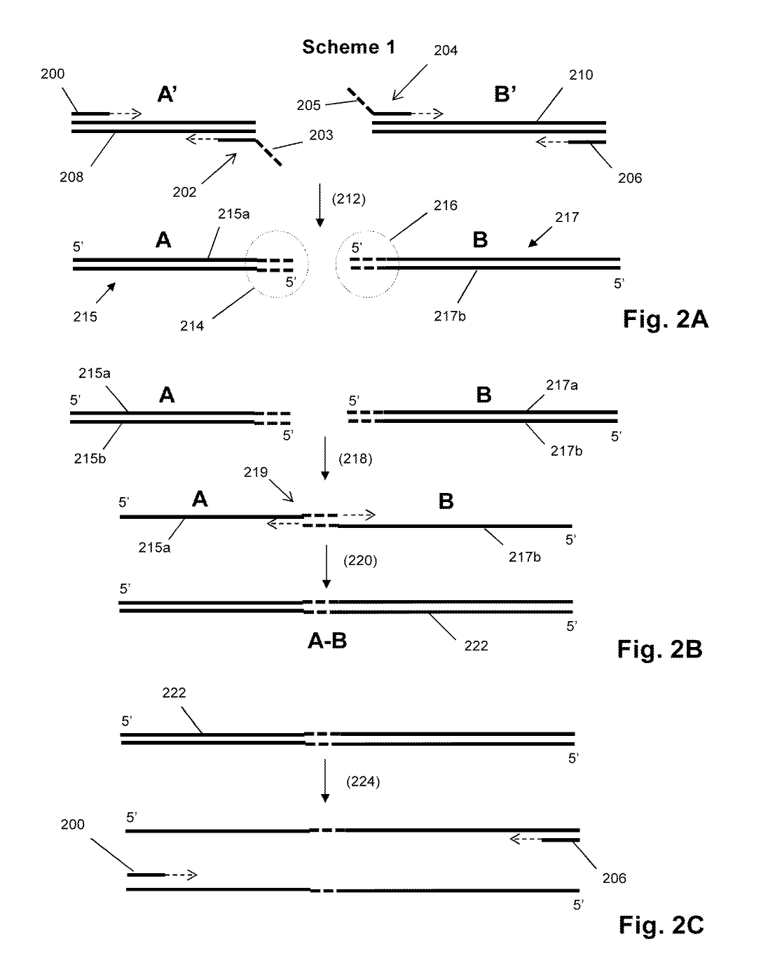

Some PCA formats useful in the present invention are described in FIGS. 2A-2C, 3A-3C, 4A-4C, 5A-5D, and 6A-6E. FIGS. 2A-2C illustrate an exemplary PCA scheme (“Scheme 1”) for joining two separate fragments A′ (208) and B′ (210) into a single fusion product (222). Fragment A′ (208) is amplified with primers (200) and (202) and fragment B′ (210) is amplified with primers (206) and (204) in the same PCR mixture. Primers (200) and (206) are “outer” primers of the PCA reaction and primers (202) and (204) are the “inner” primers of the PCA reaction. Inner primers (202) and (204) each have a tail (203 and 205, respectively) that are not complementary to A′ or B′ (or adjacent sequences if A′ and B′ are segments imbedded in a longer sequence). Tails (203) and (205) are complementary to one another. Generally, such inner primer tails are selected for selective hybridization to its corresponding inner primer (and not elsewhere); but otherwise such tails may vary widely in length and sequence. In one aspect, such tails have a length in the range of from 8 to 30 nucleotides; or a length in the range of from 14 to 24 nucleotides. As the PCRs progress (212), product fragments A (215) and B (217) are produced that incorporate tails (203) and (205) into end regions (214) and (216), respectively. During the PCRs product fragments A (215) and B (217) will denature and some of the “upper” strands (215a) of A anneal (218) to lower strands (217b) of B and the 3′ ends are extended (219) to form (220) fusion product A-B (222). Fusion product A-B (222) may be further amplified by an excess of outer primers (200) and (206). In some embodiments, the region of fusion product (222) formed from tails (203) and (205) may include one or more primer binding sites for use in later analysis, such as high-throughput sequencing.

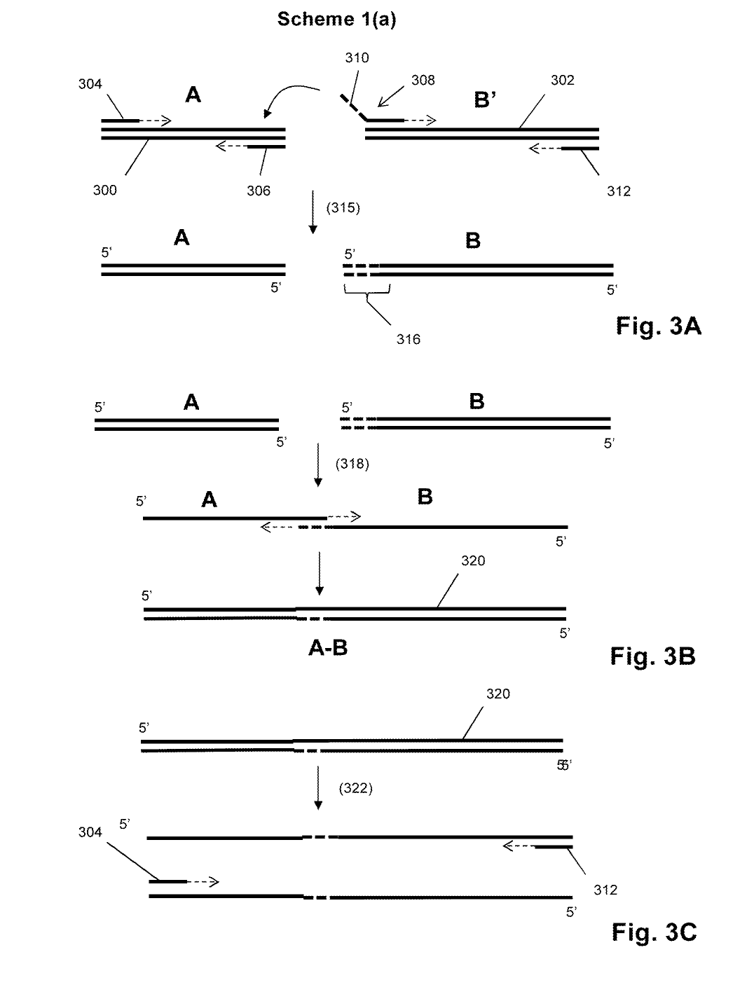

A variation of Scheme 1 is illustrated in FIGS. 3A-3C as Scheme 1(a). As above, fragment A (300) is amplified using primers (304) and (306) and fragment B′ (302) is amplified using primers (308) and (312) in PCRs carried out in a common reaction mixture. Outer primers (304) and (312) are employed as above, and inner primer (308) has tail (310); however, instead of tail (310) being complementary to a corresponding tail on primer (306), it is complementary to a segment on the end of fragment A, namely, the same segment that primer (306) is complementary to. The PCRs produce (315) fragments A and B, where B is identical to B′ (302) with the addition of segment (316) created by tail (310) of primer (308). As above, as temperature cycling continues (particularly as inner primers become exhausted), the upper fragments of fragment A anneal (318) to the lower fragment of fragment B and are extended to produce fusion product A-B (320), which may be further amplified using primers (304) and (312).

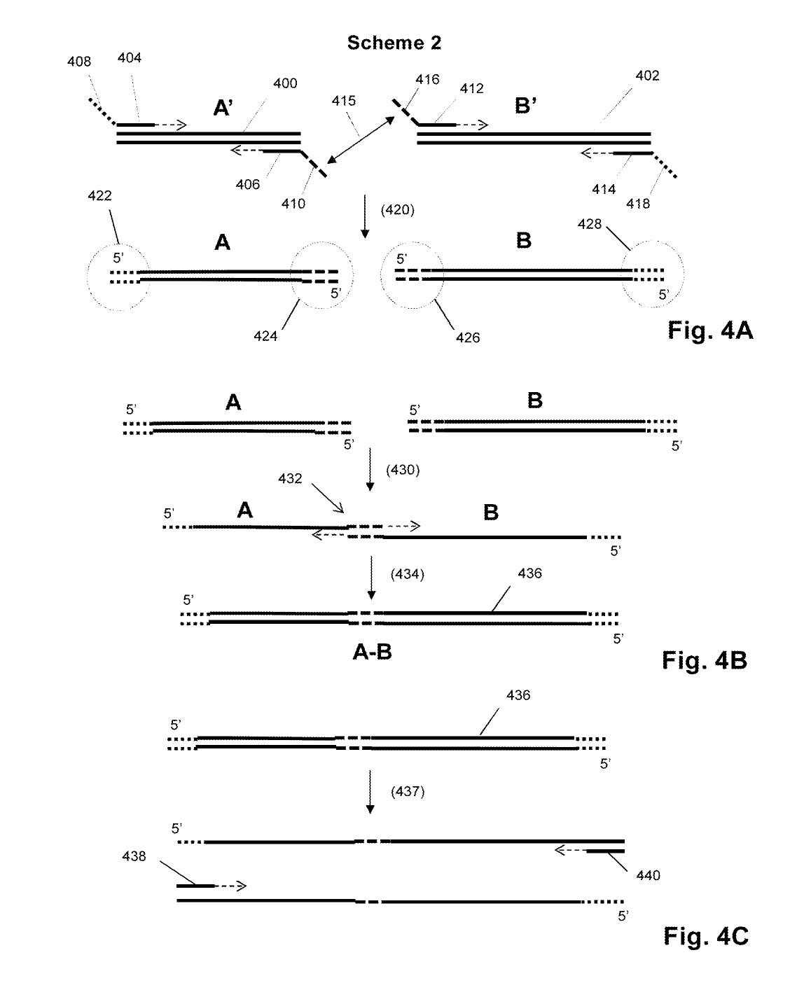

Another embodiment of a PCA that may be used with the invention (“Scheme 2”) is illustrated in FIGS. 4A-4C. The embodiment is similar to that of FIGS. 2A-2C, except that outer primers (404) and (414) have tails (408) and (418), respectively, which permit further amplification of a fusion product with predetermined primers. As discussed more fully below, this embodiment is well-suited for multiplexed amplifications. Fragment A′ (400) is amplified with primers (404) and (406), having tails (408) and (410), respectively, to produce fragment A, and fragment B′ (402) is amplified with primers (412) and (414), having tails (416) and (418), respectively, to produce (420) fragment B. Tails (410 and 416) of inner primers (406 and 412) are selected to complementary (415) to one another. Ends of fragments A and B are augmented by segments (422, 424, 426 and 428) generated by tails (408, 410, 416 and 418, respectively). As with previously described embodiments, upper strands of fragment A anneal (430) to lower strands of fragment B and are extended (432) to form (434) fusion product A-B (436) that may be further amplified (437) using primers (438 and 440) that are the same as primers (404 and 414), but without tails.

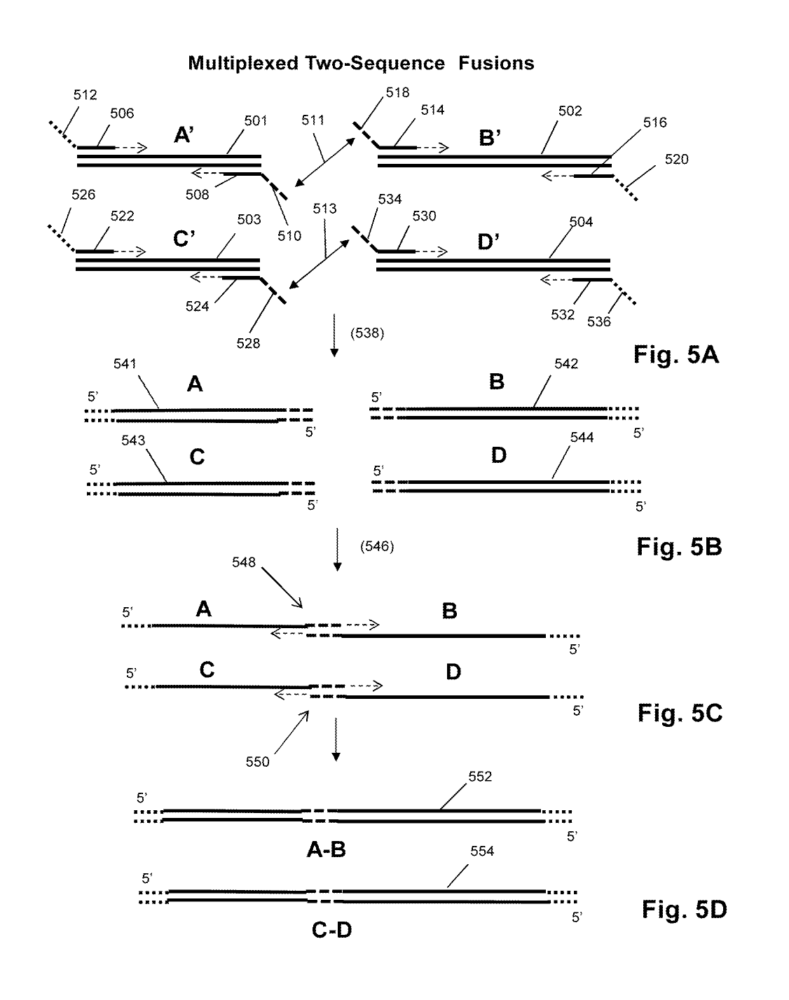

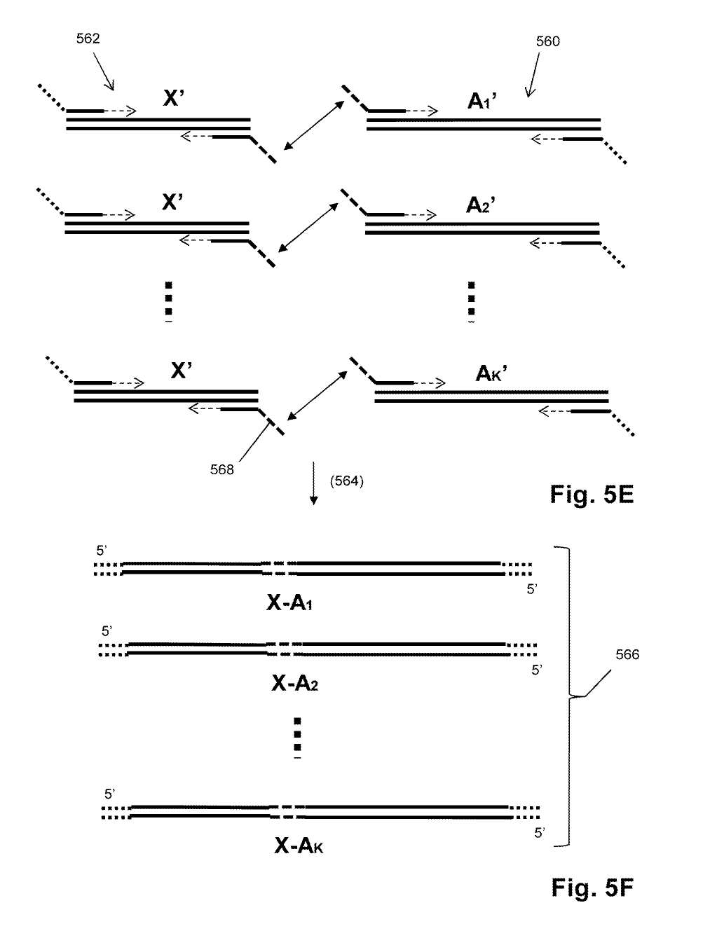

As mentioned above, the embodiment of FIGS. 4A-4C, may be used in a multiplex PCA reaction, which is illustrated in FIGS. 5A-5D. There fragments A′ (501), B′ (502), C′ (503), and D′ (504) are amplified in PCRs in a common reaction mixture using primer sets (506 and 508) for fragment A′, (514 and 516) for fragment B′, (522 and 524) for C′, and (530 and 532) for D′. All primers have tails: outer primers (506, 516, 522 and 532) each have tails (512, 520, 526 and 536, respectively) that permit both fragment amplification and subsequent fusion product amplification. Sequences of tails (512) and (520) may be the same or different from the sequences of tails (526) and (536), respectively. In one embodiment, the sequences of tails (512, 520, 526 and 536) are the same. Tails of inner primers (518 and 510) are complementary (511) to one another; likewise, tails of inner primers (528 and 534) are complementary (513) to one another. The above PCRs generate fragments A (541), B (542), C (543) and D (544), which further anneal (546) to one another to form complexes (548 and 550) which are extended to form fusion products A-B (552) and C-D (554), respectively.

FIGS. 5E and 5F illustrate a generalization of the above embodiment in which multiple different target nucleic acids (560), A₁′, A₂′, . . . A_{K}′, are linked to the same target nucleic acid, X′ (562) to form (564) multiple fusion products X-A₁, X-A₂, X-A_{K} (566). This embodiment is of particular interest when target nucleic acid, X, is a segment of recombined sequence of a lymphocyte, which can be used as a tag for the lymphocyte that it originates from. In one aspect, X is a clonotype, such as a segment of a V(D)J region of either a B cell or T cell. In one embodiment, a plurality of target nucleic acids, A₁, A₂, . . . A_{K}, are fused to the clonotype of its cell of origin. In another embodiment, such plurality is between 2 and 1000; and in another embodiment, it is between 2 and 100; and in another embodiment, it is between 2 and 10. In PCA reactions of these embodiments, the concentration of inner primer (568) may be greater than those of inner primers of the various A, nucleic acids so that there is adequate quantities of the X amplicon to anneal with the many stands of the A, amplicons. Fusion products (566) are extracted from the reaction mixture (e.g. via conventional double stranded DNA purification techniques, such as available from Qiagen, or the like) and sequenced. The sequences of the outer primers may be selected to permit direct use for cluster formation without further manipulation for sequencing systems such as a Genome Analyzer (Illumina, San Diego, Calif.). In one aspect, X may be a clonotype (for lymphocytes) or comprise a sequence tag and A₁, A₂, . . . A_{K} may be particular genes or transcripts of interest. After sequencing fusion products, per cell gene expression levels may be tabulated and/or plotted as shown in FIG. 1B.

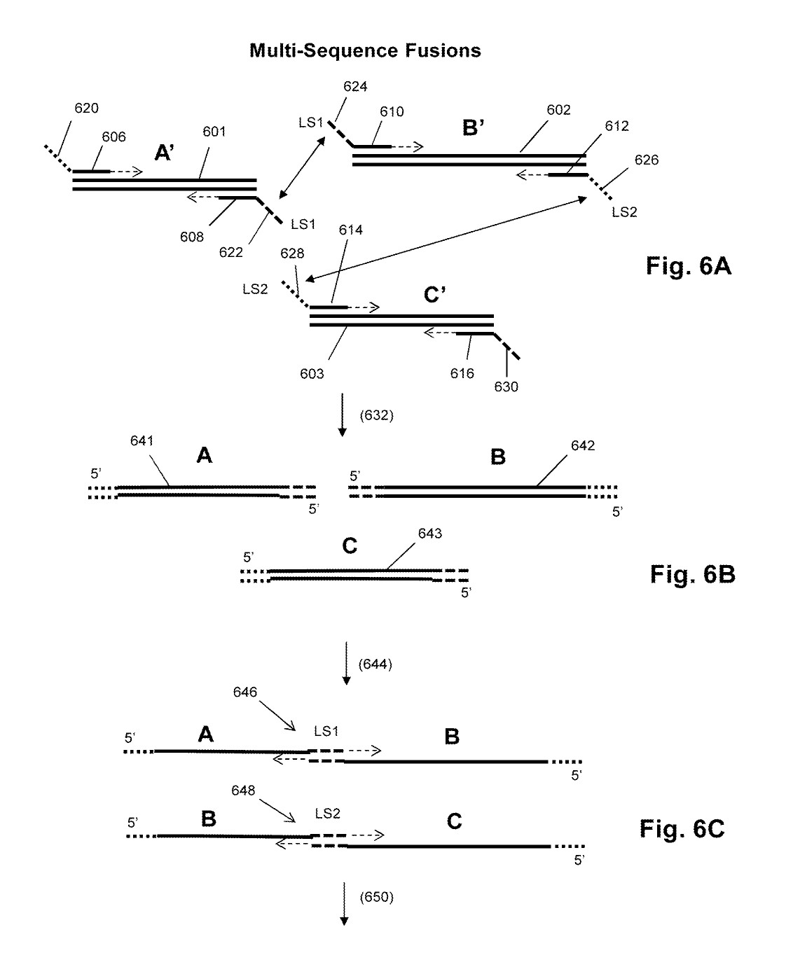

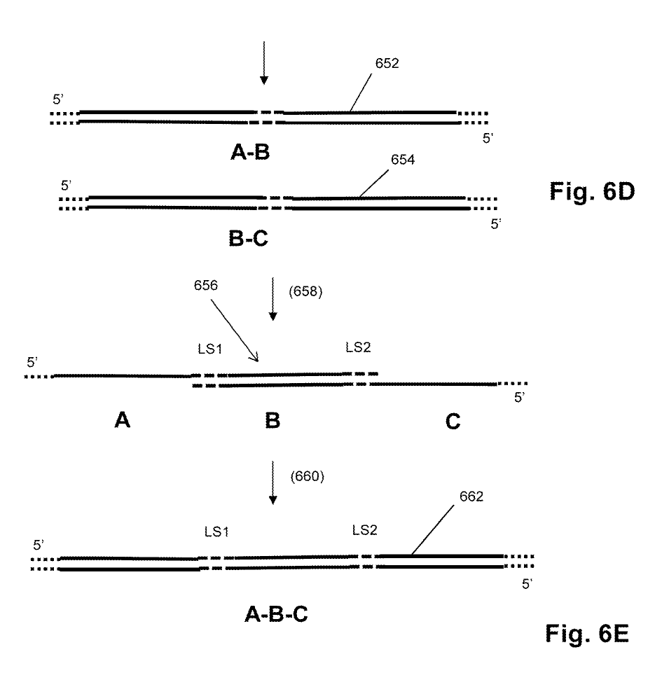

In addition to multiplexed PCA reactions in a parallel sense to simultaneously generate multiple binary fusion products, as illustrated in FIGS. 6A-6E, PCA reactions may be multiplexed in a serial sense to assemble multi-subunit fusion products. As shown in FIG. 6A, fragments A′ (601), B′ (602) and C′ (603) are amplified in a common PCR mixture with primer sets (606 and 608) for A′, (610 and 612) for B′ and (614 and 616) for C′. All primers have tails: (i) tails (620 and 630) of outer primers (606 and 616) are selected for amplification of outer fragments A′ and C′ and further amplification of three-way fusion product A-B-C (662) shown in FIG. 6E; (ii) tails (622 and 624) of inner primers (608 and 610) are complementary to one another; and (iii) tails (628 and 626) of inner primers (614 and 612) are complementary to one another. The PCRs generate (632) fragments A (641), B (642) and C (643), which in the reaction form (644) complexes (646 and 648) comprising segments LS1 and LS2, respectively, which in turn are extended to form (650) fusion products A-B (652) and B-C (654). These fusion products are denatured and some cross anneal (658) to one another by way of the common B fragment (656) to form a complex which is extended (660) to form fusion product A-B-C (662).

### Making Fusion Products Using Flap Endonuclease Reaction

In some embodiments, fusion products comprising a sequence tag and a target nucleic acid may be produced using a flap endonuclease reaction as illustrated in FIG. 1I. After reactors are formed with a single cell and single homogeneous sequence tag, conditions are adjusted (e.g. temperature raised to activate a tag-releasing endonuclease) so that molecules (1102) are produced in each reactor. Each molecule (1102) comprises primer binding site (1101), sequence tag (1103) (unique to the reactor), and segment (1105) that is capable of annealing to oligonucleotides (1104), each of which comprises a portion (1109) specific to a target polynucleotide, e.g. (1107) Oligonucleotides (1104) are referred to herein as “helper oligonucleotides.” With the release of molecules (1102) from the homogeneous sequence tag, a flap structure (1111) forms comprising a molecule (1102), an oligonucleotide (1104) and target nucleic acid (1107). Conditions are selected so that in the presence of a flap endonuclease flap structure (1111) is cleaved releasing a 5′ portion (1113) of target nucleic acid (1107) and leaving an end that may be ligated (1114) to the 3′ end of molecule (1102) of flap structure (1111). Upon ligation (1114) fusion product (1115) is formed that may be amplified (1116) by implementing a PCR in the presence of primer (1106) specific for primer binding site (1101) and primers (1108) specific for selected sites on the target nucleic acids.

FIG. 1I shows reagents for embodiments illustrated in FIG. 1H. Reagents common to all micelles formed as part of a reaction include (i) primer (1117) specific for primer binding site (1101) of sequence tag-containing molecules (1122) (also referred to as 1102 in FIG. 1H), (ii) molecules (1122) which are released from a homogeneous sequence tag and which contain sequence tag (1103) unique to a reactor, (iii) oligonucleotides (1118) (o₁, o₂ . . . oₖ in FIG. 1I and also referred to collectively as 1104 in FIG. 1H, or as helper oligonucleotides) which each comprise a 5′ portion (1109) specific for a target nucleic acid and a 3′ portion specific for portion (1105) of molecule (1122) to form flap structure (1111) for each different target nucleic acid, and (iv) target nucleic acid-specific primers (1119) (p₁, p₂ . . . pₖ in FIG. 1I and also referred to collectively as (1108) in FIG. 1H).

Flap endonucleases for carrying out the above reactions are disclosed in the following references that are incorporated herein by reference: U.S. Pat. No. 6,255,081; Matsui et al, J. Biol. Chem., 274 (26): 18297-18309 (1999); Olivier, Mutation Research, 573: 103-110 (2005); Fors et al, Pharmacogenomics, 9(1): 37-47 (1999); and the like.

In one aspect, the above embodiment may be carried out using the following steps: (a) providing multiple reactors each containing a single cell of the population, a first homogeneous sequence tag and a second homogeneous sequence tag in an amplification mixture, the amplification mixture comprising a pair of primers for amplifying each target nucleic acid of the plurality; (b) providing amplifiable sequence tags from the homogeneous sequence tags in the presence of helper oligonucleotides so that flap structures form at 5′ ends of strands of the target nucleic acids, wherein the helper oligonucleotide of each flap structure comprises a 5′ portion complementary to a strand of a target nucleic acid and a 3′ portion complementary to an amplifiable sequence tag or a product thereof; (c) cleaving the flap structures with a flap endonuclease to provide 5′ ends on the strands of target nucleic acids that are ligatable to amplifiable sequence tags; (d) ligating the amplifiable sequence tags to the ligatable 5′ ends of the strands of target nucleic acids of each flap structure; (e) amplifying the strands of each target nucleic acid and amplifiable sequence tags to form amplicons comprising sequence tags; and (f) sequencing the amplicons from the reactors to identify the target nucleic acids of each cell from the population by the sequence tags incorporated into the amplicons.

### Random Genomic Segment as a Homogeneous Sequence Tag

In some embodiments, a homogeneous sequence tag comprises a random segment of genomic DNA of the cell to be identified or a random segment of a transcriptome of the cell to be identified. In some embodiments, “transcriptome” means the total set of transcripts present in a cell; in some embodiments, “transcriptome” means the total set of transcripts present in the cytoplasm of a cell. In some embodiments, an RNA transcriptome is converted into DNA by a step of reverse transcribing the transcriptome by a reverse transcriptase. In further embodiments, such random segment is generated by digestion of cellular DNA by a subset of restriction endonucleases having an interrupted palindrome recognition sequence. The enzymes of this subset are referred to herein as “site-excision” restriction endonucleases, and they are characterized by the following properties: (i) interrupted palindromic recognition sequence, (ii) two excision sites, one of which is upstream of the recognition sequence and the other of which is downstream of the recognition sequence, and (iii) production of an excised sequence of a defined length that contains the recognition site. Exemplary site-excision restriction endonucleases are as follows:

| Name | Recognition Sequence* |
|---|---|
| Alfl | (10/12)GCANNNNNNTGC(12/10) |
| Bdal | (10/12)TGANNNNNNTCA(12/10) |
| Bpll | (8/13)GAGNNNNNCTC(13/8) |
| Fall | (8/13)AAGNNNNNCTT(13/8) |

*New England Biolab's naming convention is followed.

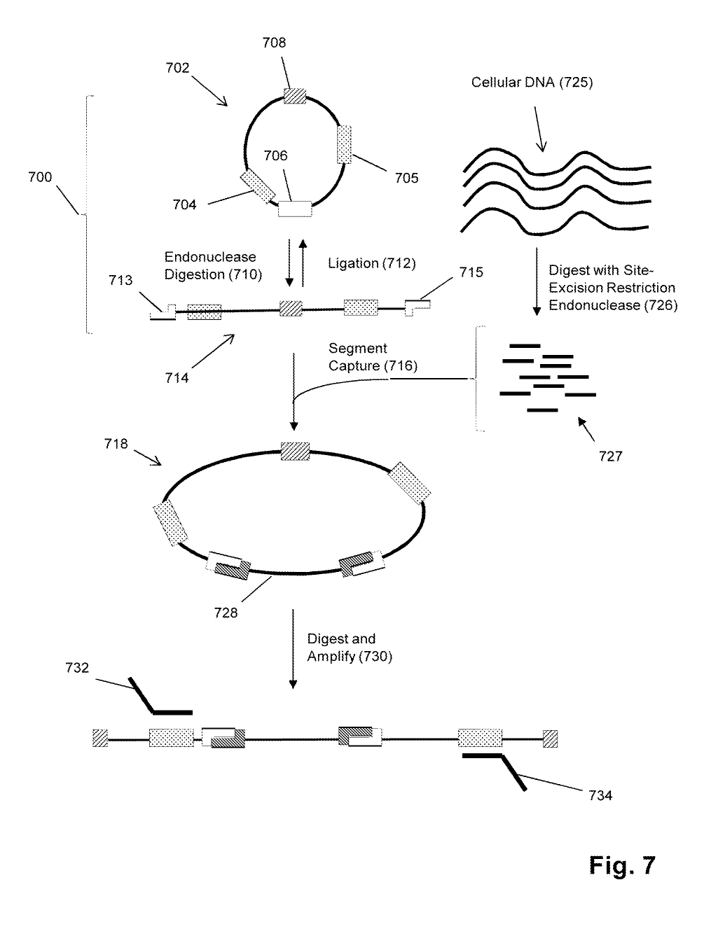

Double stranded DNA (dsDNA) circle (702) is provided with a restriction endonuclease activity recognizing recognition site (706) and a ligase activity so that an equilibrium (700) exists between the circularized state (702) and linear state (714) of the molecule (FIG. 7). Whenever dsDNA circle (702) is thus provided in a single copy, it exists alternatively in circular form (702) and in linear form (714). Endonuclease activity (710) cleaves dsDNA circle (702) to produce linear dsDNA molecule (714) and ligation activity (712) catalyzes re-formation of phosphodiester bonds between ends (713) and (715). In accordance with this embodiment of the invention, dsDNA circle (702) in a reaction mixture is provided to reactors (such as, micelles in an emulsion) in a concentration so that each reactor of a portion of the reactors contains only one dsDNA circle (702). dsDNA circle (702) includes primer binding sites (704) and (705) and optionally second restriction endonuclease recognition site (706), which for example, may recognized by a thermal stable endonuclease for linearizing construct (718) for latter amplification. In the same reactor, cellular DNA (725) is digested with site-excision restriction endonuclease (726) to produce variable length strands (not shown) and excision products (727). After incubation, circular DNA product (718) forms comprising DNA from circle (702) and random fragment (728) which will serve as a sequence tag. After digestion (730) of dsDNA circle via restriction site (708), the resulting linear construct may be conjugated with target polynucleotide of interest by way of a PCA reaction as describe above, for example, using common primers (732) and (734) specific for primer binding sites (704) and (705).

### Multiple Sequence Tags Per Reactor

In some embodiments, more than one sequence tag may be used in reactors containing a single cell. For example, in some embodiments, reactors or micelles may be selected that each contain a first homogeneous sequence tag that releases sequence tags that are attached to one strand of a double stranded target nucleic acid and a second homogeneous sequence tag that releases sequence tags that are attached to the other strand of a double stranded target nucleic acid. Such embodiments may be based on PCRs or flap endonuclease reactions as described above. For example, FIG. 1J illustrates a two-sequence tag embodiment employing a flap endonuclease reaction. Emulsion (1230) is generated containing a portion of micelles (e.g. 1231) with first homogeneous sequence tags and a single cell, a portion of micelles (e.g. 1233) with second homogeneous sequence tags and a single cell, and a portion of micelles (e.g. 1235) with first and second homogeneous sequence tags and a single cell. Flap endonuclease reaction (1232) is illustrated below for one target nucleic acid (1218) of a micelle (1235) that contains first and second homogeneous sequence tags. Conditions are selected so that target nucleic acid (1218) denatures into strand Si (1220) and its complement Si′ (1221), after which both stands combine with their respective reaction elements to form first flap structure (1224) and second flap structure (1226). In the presence of a flap endonuclease and a ligase, a unique sequence tag (1225) is attached to strand Si (1220) and a different unique sequence tag (1227) is attached to its complement Si′ (1221). The resulting fusion products may be further amplified (1240) in a PCR.

### Single Cell Analysis

As mentioned above, in one aspect of the invention, cells from a population are disposed in reactors each containing a single cell. This may be accomplished by a variety of large-scale single-cell reactor platforms known in the art, e.g. Clarke et al, U.S. patent publication 2010/0255471; Mathies et al, U.S. patent publication 2010/0285975; Edd et al, U.S. patent publication 2010/0021984; Colston et al, U.S. patent publication 2010/0173394; Love et al, International patent publication WO2009/145925; Muraguchi et al, U.S. patent publication 2009/0181859; Novak et al, Angew. Chem. Int. Ed., 50: 390-395 (2011); Chen et al, Biomed Microdevices, 11: 1223-1231 (2009); and the like, which are incorporated herein by reference. In one aspect, cells are disposed in wells of a microwell array where reactions, such as PCA reactions, take place; in another aspect, cells are disposed in micelles of a water-in-oil emulsion, where micelles serve as reactors. Micelle reactors generated by microfluidics devices, e.g. Mathies et al (cited above) or Edd et al (cited above), are of particular interest because uniform-sized micelles may be generated with lower shear and stress on cells than in bulk emulsification processes. Compositions and techniques for emulsifications, including carrying out amplification reactions, such as PCRs, in micelles is found in the following references, which are incorporated by reference: Becher, “Emulsions: Theory and Practice,” (Oxford University Press, 2001); Griffiths and Tawfik, U.S. Pat. No. 6,489,103; Tawfik and Griffiths, Nature Biotechnology, 16: 652-656 (1998); Nakano et al, J. Biotechnology, 102: 117-124 (2003); Dressman et al, Proc. Natl. Acad. Sci., 100: 8817-8822 (2003); Dressman et al, U.S. Pat. No. 8,048,627; Berka et al, U.S. Pat. Nos. 7,842,457 and 8,012,690; Diehl et al, Nature Methods, 3: 551-559 (2006); Williams et al, Nature Methods, 3: 545-550 (2006); Zeng et al, Analytical Chemistry, 82(8): 3183-3190 (2010); Micellula DNA Emulsion & Purification Kit instructions (EURx, Gdansk, Poland, 2011); and the like. In one embodiment, the mixture of homogeneous sequence tags (e.g. beads) and reaction mixture is added dropwise into a spinning mixture of biocompatible oil (e.g., light mineral oil, Sigma) and allowed to emulsify. In another embodiment, the homogeneous sequence tags and reaction mixture are added dropwise into a cross-flow of biocompatible oil. The oil used may be supplemented with one or more biocompatible emulsion stabilizers. These emulsion stabilizers may include Atlox 4912, Span 80, and other recognized and commercially available suitable stabilizers. In some embodiments, the emulsion is heat stable to allow thermal cycling, e.g., to at least 94° C., at least 95° C., or at least 96° C. Preferably, the droplets formed range in size from about 5 microns to about 500 microns, more preferably from about 10 microns to about 350 microns, even more preferably from about 50 to 250 microns, and most preferably from about 100 microns to about 200 microns. Advantageously, cross-flow fluid mixing allows for control of the droplet formation, and uniformity of droplet size.

In some embodiments, micelles are produced having a uniform distribution of volumes so that reagents available in such reactors result in similarly amplified target nucleic acids and sequence tags. That is, widely varying reactor volumes, e.g. micelle volumes, may lead to amplification failures and/or widely varying degrees of amplification. Such failures and variation would preclude or increase the difficulty of making quantitative comparisons of target nucleic acids in individual cells of a population, e.g. differences in gene expression. In one aspect, micelles are produced that have a distribution of volumes with a coefficient of variation (CV) of thirty percent or less. In some embodiments, micelles have a distribution of volumes with a CV of twenty percent of less.

Cells of a sample and homogeneous sequence tags may be suspended in a reaction mixture prior to disposition into reactors. In one aspect, a reaction mixture is a PCA reaction mixture and is substantially the same as a PCR reaction mixture with at least one pair of inner (or linking) primers and at least one pair of outer primers. A reaction mixture may comprise one or more optional components, including but not limited to, thermostable restriction endonucleases to release sequence tagged primers from a homogeneous sequence tag; one or more proteinase inhibitors; lysing agents to facilitate release of target nucleic acids of isolated cells, e.g. Brown et al, Interface, 5: S131-S138 (2008); and the like. In some embodiments, a step of lysing cells may be accomplished by heating cells to a temperature of 95° C. or above in the presence of a nonionic detergent, e.g. 0.1% Tween X-100, for a period prior to carrying out an amplification reaction. In one embodiment, such period of elevated temperature may be from 10-20 minutes. Alternatively, a step of lysing cells may be accomplished by one or more cycles of heating and cooling, e.g. 96° C. for 15 min followed by 10° C. for 10 min, in the presence of a nonionic detergent, e.g. 0.1% Tween X-100.

In some embodiments, micelle reactors are generated and sorted in a microfluidics device, such as illustrated in FIG. 1K, many features of which are disclosed in Chen et al (cited above), which is incorporated by reference. Aqueous reaction mixture (1306) containing cells (1302) and homogeneous sequence tags (1304) are provided in reservoir (1300) in concentrations to ensure formation of micelles containing a single cell and a single homogeneous sequence tag under selected operating conditions. Reaction mixture (1306) flows through passage (1305) into junction (1307) where it meets oil flows from passages (1308) and (1309). The flow rates and pressures of the three flows are adjusted so that aqueous micelles are formed in junction (1307) and are carried by combined oil flows from passages (1308) and (1309) through passage (1311) and eventually pass through interrogation region (1312), where the presence, absence or level of one or more predetermined characteristics of each micelles is determined. Predetermined characteristics may include the presence or absence of a cell or particle in a micelle and the presence or absence of one or more homogeneous sequence tags in a micelle. In some embodiments, detection of such characteristics may be carried out using distinct fluorescent probes specifically bound to homogeneous sequence tags and/or to cells. For example, one or more fluorescently labeled antibodies with first emission characteristics may label cells and one or more fluorescently labeled oligonucleotide probes with second emission characteristics may label homogeneous sequence tags. Detectors associated with interrogation region (1312) are operationally associated with an effector region (1313) where a force is applied to a micelle when it reaches effector region (1313) based on the signals detected in interrogation region (1312). Force to direct a micelle to alternative flows through different passages may be acoustic, optical, or the like. In one embodiment, an acoustic force (1314) is applied in accordance with the teaching in Chen et al (cited above) to direct micelles (1320) containing both a single cell and a single homogeneous sequence tag into passage 3 (1342), micelles (1316) containing only one or more cells into passage 1 (1344), and remaining micelles (1318) to passage 2 (1346).

Clearly many other microfluidics device configurations may be employed to generate micelles containing a single cell and a predetermined number of homogeneous sequence tags, for example, one homogeneous sequence tag, two homogeneous sequence tags, or to selectively add reagents to a micelle by selectively coalescing micelles, by electroporation, or the like, e.g. Zagoni et al, chapter 2, Methods of Cell Biology, 102: 25-48 (2011); Brouzes, chapter 10, Methods of Cell Biology, 102: 105-139 (2011); Wiklund et al, chapter 14, Methods of Cell Biology, 102: 177-196 (2011); Le Gac et al, chapter 7, Methods of Molecular Biology, 853: 65-82 (2012); and the like.

### Nucleic Acid Sequencing Techniques

Any high-throughput technique for sequencing nucleic acids can be used in the method of the invention. DNA sequencing techniques include dideoxy sequencing reactions (Sanger method) using labeled terminators or primers and gel separation in slab or capillary, sequencing by synthesis using reversibly terminated labeled nucleotides, pyrosequencing, 454 sequencing, sequencing by synthesis using allele specific hybridization to a library of labeled clones that is followed by ligation, real time monitoring of the incorporation of labeled nucleotides during a polymerization step, polony sequencing, SOLiD sequencing, and the like. These sequencing approaches can thus be used to sequence fusion products of target nucleic acids of interest and clonotypes based on T-cell receptors (TCRs) and/or B-cell receptors (BCRs). In one aspect of the invention, high-throughput methods of sequencing are employed that comprise a step of spatially isolating individual molecules on a solid surface where they are sequenced in parallel. Such solid surfaces may include nonporous surfaces (such as in Solexa sequencing, e.g. Bentley et al, Nature, 456: 53-59 (2008) or Complete Genomics sequencing, e.g. Drmanac et al, Science, 327: 78-81 (2010)), arrays of wells, which may include bead- or particle-bound templates (such as with 454, e.g. Margulies et al, Nature, 437: 376-380 (2005) or Ion Torrent sequencing, U.S. patent publication 2010/0137143 or 2010/0304982), micromachined membranes (such as with SMRT sequencing, e.g. Eid et al, Science, 323: 133-138 (2009)), or bead arrays (as with SOLiD sequencing or polony sequencing, e.g. Kim et al, Science, 316: 1481-1414 (2007)). In another aspect, such methods comprise amplifying the isolated molecules either before or after they are spatially isolated on a solid surface. Prior amplification may comprise emulsion-based amplification, such as emulsion PCR, or rolling circle amplification. Of particular interest is Solexa-based sequencing where individual template molecules are spatially isolated on a solid surface, after which they are amplified in parallel by bridge PCR to form separate clonal populations, or clusters, and then sequenced, as described in Bentley et al (cited above) and in manufacturer's instructions (e.g. TruSeq™ Sample Preparation Kit and Data Sheet, Illumina, Inc., San Diego, Calif., 2010); and further in the following references: U.S. Pat. Nos. 6,090,592; 6,300,070; 7,115,400; and EP0972081B1; which are incorporated by reference. In one embodiment, individual molecules disposed and amplified on a solid surface form clusters in a density of at least 10⁵ clusters per cm²; or in a density of at least 5×10⁵ per cm²; or in a density of at least 10⁶ clusters per cm². In one embodiment, sequencing chemistries are employed having relatively high error rates. In such embodiments, the average quality scores produced by such chemistries are monotonically declining functions of sequence read lengths. In one embodiment, such decline corresponds to 0.5 percent of sequence reads have at least one error in positions 1-75; 1 percent of sequence reads have at least one error in positions 76-100; and 2 percent of sequence reads have at least one error in positions 101-125.

In some embodiments, multiplex PCR is used to amplify members of a mixture of nucleic acids, particularly mixtures comprising recombined immune molecules such as T cell receptors, B cell receptors, or portions thereof. Guidance for carrying out multiplex PCRs of such immune molecules is found in the following references, which are incorporated by reference: Morley, U.S. Pat. No. 5,296,351; Gorski, U.S. Pat. No. 5,837,447; Dau, U.S. Pat. No. 6,087,096; Von Dongen et al, U.S. patent publication 2006/0234234; European patent publication EP 1544308B1; Faham et al, U.S. patent publication 2010/0151471; Han, U.S. patent publication 2010/0021896; Robins et al, U.S. patent publication 2010/033057; and the like. Such amplification techniques are readily modified by those of ordinary skill in the art to supply outer primers and linking primers of the invention.

While the present invention has been described with reference to several particular example embodiments, those skilled in the art will recognize that many changes may be made thereto without departing from the spirit and scope of the present invention. The present invention is applicable to a variety of sensor implementations and other subject matter, in addition to those discussed above.

### DEFINITIONS

Unless otherwise specifically defined herein, terms and symbols of nucleic acid chemistry, biochemistry, genetics, and molecular biology used herein follow those of standard treatises and texts in the field, e.g. Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Abbas et al, Cellular and Molecular Immunology, 6th edition (Saunders, 2007).

“Amplicon” means the product of a polynucleotide amplification reaction; that is, a clonal population of polynucleotides, which may be single stranded or double stranded, which are replicated from one or more starting sequences. The one or more starting sequences may be one or more copies of the same sequence, or they may be a mixture of different sequences. In some embodiments, amplicons are formed by the amplification of a single starting sequence. Amplicons may be produced by a variety of amplification reactions whose products comprise replicates of the one or more starting, or target, nucleic acids. In one aspect, amplification reactions producing amplicons are “template-driven” in that base pairing of reactants, either nucleotides or oligonucleotides, have complements in a template polynucleotide that are required for the creation of reaction products. In one aspect, template-driven reactions are primer extensions with a nucleic acid polymerase or oligonucleotide ligations with a nucleic acid ligase. Such reactions include, but are not limited to, polymerase chain reactions (PCRs), linear polymerase reactions, nucleic acid sequence-based amplification (NASBAs), rolling circle amplifications, and the like, disclosed in the following references that are incorporated herein by reference: Mullis et al, U.S. Pat. Nos. 4,683,195; 4,965,188; 4,683,202; 4,800,159 (PCR); Gelfand et al, U.S. Pat. No. 5,210,015 (real-time PCR with “taqman” probes); Wittwer et al, U.S. Pat. No. 6,174,670; Kacian et al, U.S. Pat. No. 5,399,491 (“NASBA”); Lizardi, U.S. Pat. No. 5,854,033; Aono et al, Japanese patent publ. JP 4-262799 (rolling circle amplification); and the like. In one aspect, amplicons of the invention are produced by PCRs. An amplification reaction may be a “real-time” amplification if a detection chemistry is available that permits a reaction product to be measured as the amplification reaction progresses, e.g. “real-time PCR” described below, or “real-time NASBA” as described in Leone et al, Nucleic Acids Research, 26: 2150-2155 (1998), and like references. As used herein, the term “amplifying” means performing an amplification reaction. A “reaction mixture” or “amplification mixture” means a solution containing all the necessary reactants for performing a reaction, which may include, but not be limited to, buffering agents to maintain pH at a selected level during a reaction, salts, co-factors, scavengers, and the like.

“Kit” refers to any delivery system for delivering materials or reagents for carrying out a method of the invention. In the context of methods of the invention, such delivery systems include systems that allow for the storage, transport, or delivery of reaction reagents (e.g., primers, enzymes, internal standards, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. Such contents may be delivered to the intended recipient together or separately. For example, a first container may contain an enzyme for use in an assay, while a second container contains primers.

“Ligation” means to form a covalent bond or linkage between the termini of two or more nucleic acids, e.g. oligonucleotide and/or polynucleotide, in a template-driven reaction. The nature of the bond or linkage may vary widely and the ligation may be carried out enzymatically or chemically. As used herein, ligations are usually carried out enzymatically to form a phosphodiester linkage between a 5′ carbon of a terminal nucleotide of one oligonucleotide with 3′ carbon of another oligonucleotide. A variety of template-driven ligation reactions are described in the following references, which are incorporated by reference: Whitely et al, U.S. Pat. No. 4,883,750; Letsinger et al, U.S. Pat. No. 5,476,930; Fung et al, U.S. Pat. No. 5,593,826; Kool, U.S. Pat. No. 5,426,180; Landegren et al, U.S. Pat. No. 5,871,921; Xu and Kool, Nucleic Acids Research, 27:875-881 (1999); Higgins et al, Methods in Enzymology, 68:50-71 (1979); Engler et al. The Enzymes. 15:3-29 (1982); and Namsaraev, U.S. patent publication 2004/0110213.

“Microfluidics device” means an integrated system of one or more chambers, ports, and channels that are interconnected and in fluid communication and designed for carrying out an analytical reaction or process, either alone or in cooperation with an appliance or instrument that provides support functions, such as sample introduction, fluid and/or reagent driving means, temperature control, detection systems, data collection and/or integration systems, and the like. Microfluidics devices may further include valves, pumps, and specialized functional coatings on interior walls, e.g. to prevent adsorption of sample components or reactants, facilitate reagent movement by electroosmosis, or the like. Such devices are usually fabricated in or as a solid substrate, which may be glass, plastic, or other solid polymeric materials, and typically have a planar format for ease of detecting and monitoring sample and reagent movement, especially via optical or electrochemical methods. Features of a microfluidic device usually have cross-sectional dimensions of less than a few hundred square micrometers and passages typically have capillary dimensions, e.g. having maximal cross-sectional dimensions of from about 500 μm to about 0.1 μm. Microfluidics devices typically have volume capacities in the range of from 1 μL to a few nL, e.g. 10-100 nL. The fabrication and operation of microfluidics devices are well-known in the art as exemplified by the following references that are incorporated by reference: Ramsey, U.S. Pat. Nos. 6,001,229; 5,858,195; 6,010,607; and 6,033,546; Soane et al, U.S. Pat. Nos. 5,126,022 and 6,054,034; Nelson et al, U.S. Pat. No. 6,613,525; Maher et al, U.S. Pat. No. 6,399,952; Ricco et al, International patent publication WO 02/24322; Bjornson et al, International patent publication WO 99/19717; Wilding et al, U.S. Pat. Nos. 5,587,128; 5,498,392; Sia et al, Electrophoresis, 24: 3563-3576 (2003); Unger et al, Science, 288: 113-116 (2000); Enzelberger et al, U.S. Pat. No. 6,960,437.

“Polymerase chain reaction,” or “PCR,” means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g. exemplified by the references: Innis et al, editors, PCR Protocols (Academic Press, 1990); McPherson et al, editors, PCR: A Practical Approach and PCR2: A Practical Approach (IRL Press, Oxford, 1991 and 1995, respectively). For example, in a conventional PCR using Tali DNA polymerase, a double stranded target nucleic acid may be denatured at a temperature >90° C., primers annealed at a temperature in the range 50-75° C., and primers extended at a temperature in the range 72-78° C. A typical amplification mixture for PCR contains at least one forward primer and at least one reverse primer in concentrations between 0.1 and 0.5 μM; dNTPs in concentrations between 100-300 μM; DNA polymerase together with salts (e.g. 10-50 mM KCl or NaCl, and 1-6 mM MgCl₂); and a buffering agent (e.g. 10-50 mM Tris-HCl at pH 8.3-8.8). Reaction volumes range from a few hundred nanoliters, e.g. 200 nL, to a few hundred μL, e.g. 200 μL. The term “PCR” encompasses derivative forms of the reaction, including but not limited to, RT-PCR, real-time PCR, nested PCR, quantitative PCR, multiplexed PCR, and the like. The particular format of PCR being employed is discernible by one skilled in the art from the context of an application. “Reverse transcription PCR,” or “RT-PCR,” means a PCR that is preceded by a reverse transcription reaction that converts a target RNA to a complementary single stranded DNA, which is then amplified, e.g. Tecott et al, U.S. Pat. No. 5,168,038, which patent is incorporated herein by reference. “Real-time PCR” means a PCR for which the amount of reaction product, i.e. amplicon, is monitored as the reaction proceeds. There are many forms of real-time PCR that differ mainly in the detection chemistries used for monitoring the reaction product, e.g. Gelfand et al, U.S. Pat. No. 5,210,015 (“taqman”); Wittwer et al, U.S. Pat. Nos. 6,174,670 and 6,569,627 (intercalating dyes); Tyagi et al, U.S. Pat. No. 5,925,517 (molecular beacons); which patents are incorporated herein by reference. Detection chemistries for real-time PCR are reviewed in Mackay et al, Nucleic Acids Research, 30: 1292-1305 (2002), which is also incorporated herein by reference. “Nested PCR” means a two-stage PCR wherein the amplicon of a first PCR becomes the sample for a second PCR using a new set of primers, at least one of which binds to an interior location of the first amplicon. As used herein, “initial primers” in reference to a nested amplification reaction mean the primers used to generate a first amplicon, and “secondary primers” mean the one or more primers used to generate a second, or nested, amplicon. “Multiplexed PCR” means a PCR wherein multiple target sequences (or a single target sequence and one or more reference sequences) are simultaneously carried out in the same reaction mixture, e.g. Bernard et al, Anal. Biochem., 273: 221-228 (1999)(two-color real-time PCR). Usually, distinct sets of primers are employed for each sequence being amplified. Typically, the number of target sequences in a multiplex PCR is in the range of from 2 to 50, or from 2 to 40, or from 2 to 30. “Quantitative PCR” means a PCR designed to measure the abundance of one or more specific target sequences in a sample or specimen. Quantitative PCR includes both absolute quantitation and relative quantitation of such target sequences. Quantitative measurements are made using one or more reference sequences or internal standards that may be assayed separately or together with a target sequence. The reference sequence may be endogenous or exogenous to a sample or specimen, and in the latter case, may comprise one or more competitor templates. Typical endogenous reference sequences include segments of transcripts of the following genes: β-actin, GAPDH, β2-microglobulin, ribosomal RNA, and the like. Techniques for quantitative PCR are well-known to those of ordinary skill in the art, as exemplified in the following references that are incorporated by reference: Freeman et al, Biotechniques, 26: 112-126 (1999); Becker-Andre et al, Nucleic Acids Research, 17: 9437-9447 (1989); Zimmerman et al, Biotechniques, 21: 268-279 (1996); Diviacco et al, Gene, 122: 3013-3020 (1992); Becker-Andre et al, Nucleic Acids Research, 17: 9437-9446 (1989); and the like.

“Primer” means an oligonucleotide, either natural or synthetic that is capable, upon forming a duplex with a polynucleotide template, of acting as a point of initiation of nucleic acid synthesis and being extended from its 3′ end along the template so that an extended duplex is formed. Extension of a primer is usually carried out with a nucleic acid polymerase, such as a DNA or RNA polymerase. The sequence of nucleotides added in the extension process is determined by the sequence of the template polynucleotide. Usually primers are extended by a DNA polymerase. Primers usually have a length in the range of from 14 to 40 nucleotides, or in the range of from 18 to 36 nucleotides. Primers are employed in a variety of nucleic amplification reactions, for example, linear amplification reactions using a single primer, or polymerase chain reactions, employing two or more primers. Guidance for selecting the lengths and sequences of primers for particular applications is well known to those of ordinary skill in the art, as evidenced by the following references that are incorporated by reference: Dieffenbach, editor, PCR Primer: A Laboratory Manual, 2nd Edition (Cold Spring Harbor Press, New York, 2003).

“Sequence read” means a sequence of nucleotides determined from a sequence or stream of data generated by a sequencing technique, which determination is made, for example, by means of base-calling software associated with the technique, e.g. base-calling software from a commercial provider of a DNA sequencing platform. A sequence read usually includes quality scores for each nucleotide in the sequence. Typically, sequence reads are made by extending a primer along a template nucleic acid, e.g. with a DNA polymerase or a DNA ligase. Data is generated by recording signals, such as optical, chemical (e.g. pH change), or electrical signals, associated with such extension. Such initial data is converted into a sequence read.

“Sequence tag” (or “tag”) or “barcode” means an oligonucleotide that is attached to a polynucleotide or template molecule and is used to identify and/or track the polynucleotide or template in a reaction or a series of reactions. A sequence tag may be attached to the 3′- or 5′-end of a polynucleotide or template or it may be inserted into the interior of such polynucleotide or template to form a linear conjugate, sometime referred to herein as a “tagged polynucleotide,” or “tagged template,” or “tag-polynucleotide conjugate,” “tag-molecule conjugate,” or the like. Sequence tags may vary widely in size and compositions; the following references, which are incorporated herein by reference, provide guidance for selecting sets of sequence tags appropriate for particular embodiments: Brenner, U.S. Pat. No. 5,635,400; Brenner and Macevicz, U.S. Pat. No. 7,537,897; Brenner et al, Proc. Natl. Acad. Sci., 97: 1665-1670 (2000); Church et al, European patent publication 0 303 459; Shoemaker et al, Nature Genetics, 14: 450-456 (1996); Morris et al, European patent publication 0799897A1; Wallace, U.S. Pat. No. 5,981,179; and the like. Lengths and compositions of sequence tags can vary widely, and the selection of particular lengths and/or compositions depends on several factors including, without limitation, how tags are used to generate a readout, e.g. via a hybridization reaction or via an enzymatic reaction, such as sequencing; whether they are labeled, e.g. with a fluorescent dye or the like; the number of distinguishable oligonucleotide tags required to unambiguously identify a set of polynucleotides, and the like, and how different must tags of a set be in order to ensure reliable identification, e.g. freedom from cross hybridization or misidentification from sequencing errors. In one aspect, sequence tags can each have a length within a range of from 2 to 36 nucleotides, or from 4 to 30 nucleotides, or from 8 to 20 nucleotides, or from 6 to 10 nucleotides, respectively. In one aspect, sets of sequence tags are used wherein each sequence tag of a set has a unique nucleotide sequence that differs from that of every other tag of the same set by at least two bases; in another aspect, sets of sequence tags are used wherein the sequence of each tag of a set differs from that of every other tag of the same set by at least three bases.

What is claimed is:

1. A method of analyzing a plurality of target nucleic acids of single cells of a population, the method comprising the steps of:
providing multiple reactors each containing a single cell of the population and a single homogeneous sequence tag in an amplification mixture, the amplification mixture comprising a pair of primers for amplifying each target nucleic acid of the plurality;
providing amplifiable sequence tags from the homogeneous sequence tags;
amplifying the target nucleic acids and amplifiable sequence tags to form amplicons comprising sequence tags; and
sequencing the amplicons from the reactors to identify the target nucleic acids of each cell from the population by the sequence tags incorporated into the amplicons.

2. The method of claim 1 wherein said step of amplifying is carried out by a polymerase chain reaction.

3. The method of 1 wherein said step of providing said amplifiable sequence tags comprises releasing said amplifiable sequence tags from said homogeneous sequence tag.

4. The method of claim 3 wherein said step of releasing said amplifiable sequence tags is carried out by cleaving said amplifiable sequence tags from said homogeneous sequence tag by a thermostable restriction endonuclease.

5. The method of claim 4 wherein each of said amplifiable sequence tags is a sequence tagged primer.

6. The method of claim 4 wherein each of said amplifiable sequence tags is a sequence tag flanked by primer binding sites and wherein said amplification mixture further comprises a pair of primers capable of amplifying said amplifiable sequence tag in a PCR.

7. The method of claim 1 wherein said step of providing said amplifiable sequence tags comprising generating said amplifiable sequence tags by an EXPAR.

8. The method of claim 1 wherein said homogeneous sequence tag is a rolling circle amplicon comprising a plurality of said sequence tagged primers.

9. The method of claim 1 wherein said homogeneous sequence tag is a bead having a plurality of sequence tagged primers attached thereto.

10. The method of claim 1 wherein said reactors are micelles of an emulsion.

11. The method of claim 10 wherein said micelles are generated in a microfluidics device.

12. The method of claim 10 wherein said micelles have a distribution of volumes with a coefficient of variation of thirty percent or less.

13. The method of claim 1 wherein said population of said single cells are from the same sample.

14. The method of claim 1 further including a step of lysing said single cells in each of said reactors prior to said step of amplifying.

15. The method of claim 1 wherein said homogeneous sequence tag comprises a random genomic segment.

16. The method of claim 1 wherein said homogeneous sequence tag comprises a random transcriptome segment.

17. A method of analyzing a plurality target nucleic acids of each cell of a population, the method comprising the steps of:
providing multiple reactors each containing a single cell and a single homogeneous sequence tag in a polymerase cycling assembly (PCA) reaction mixture, the homogeneous sequence tag comprising at least one sequence tagged primer, and the PCA reaction mixture comprising a pair of outer primers and one or more pairs of linking primers specific for the plurality of target nucleic acids, wherein at least one of the outer primers or linking primers is a sequence tagged primer of the homogeneous sequence tag;
performing a PCA reaction in the reactors so that homogeneous sequence tags release or produce sequence tagged primers and so that fusion products of the target nucleic acids and sequence tagged primers are formed in the reactors; and
sequencing the fusion products from the reactors to identify the target nucleic acids of each cell in the population.

18. The method of claim 17 wherein said multiple reactors are aqueous micelles of a water-in-oil emulsion.

19. The method of claim 18 wherein said water-in-oil emulsion is generated by a microfluidics device.

20. The method of claim 17 wherein said target nucleic acids are transcripts of a transcriptome.

21. The method of claim 17 wherein said homogeneous sequence tag is a bead having a plurality of sequence tagged primers attached thereto.

22. The method of claim 17 further including a step of lysing said single cells in each of said reactors prior to said step of amplifying.

23. A method of analyzing a plurality of target nucleic acids of single cells of a population, the method comprising the steps of:
providing multiple reactors each containing a single cell of the population, a first homogeneous sequence tag and a second homogeneous sequence tag in an amplification mixture, the amplification mixture comprising a pair of primers for amplifying each target nucleic acid of the plurality;
providing amplifiable sequence tags from the homogeneous sequence tags in the presence of helper oligonucleotides so that flap structures form at 5′ ends of strands of the target nucleic acids;
cleaving the flap structures with a flap endonuclease to provide 5′ ends on the strands of target nucleic acids that are ligatable to amplifiable sequence tags;
ligating the amplifiable sequence tags to the ligatable 5′ ends of the strands of target nucleic acids;
amplifying the strands of each target nucleic acid and amplifiable sequence tags to form amplicons comprising sequence tags; and
sequencing the amplicons from the reactors to identify the target nucleic acids of each cell from the population by the sequence tags incorporated into the amplicons.

24. The method of claim 23 wherein said multiple reactors are aqueous micelles of a water-in-oil emulsion.

25. The method of claim 24 wherein said water-in-oil emulsion is generated by a microfluidics device.

26. The method of claim 23 wherein said target nucleic acids are transcripts of a transcriptome.

27. The method of claim 23 wherein said homogeneous sequence tag is a bead having a plurality of sequence tagged primers attached thereto.

28. The method of claim 23 further including a step of lysing said single cells in each of said reactors prior to said step of amplifying.
